(19) United States
(12) Patent Application Publication
(10) Pub. No.: US 2021/0137072 A1
(43) Pub. Date: May 13, 2021

(54) Ear Protection System for a Terrestrial Animal

(71) Applicant: Derek Beck, Martinez, CA (US)

(72) Inventor: Derek Beck, Martinez, CA (US)

(21) Appl. No.: 16/682,465

(22) Filed: Nov. 13, 2019

(51) Int. Cl. A01K 13/00; A61F 11/14; H04R 1/10

(52) U.S. Cl. CPC A01K 13/006; H04R 1/1083; A61F 11/14

(57) ABSTRACT

A cranial protection system for a terrestrial animal such as a service dog is disclosed. The cranial protection system comprises a helmet that is secured to the head over ears of the animal and a first ear protector and a second ear protector, wherein each one of the first ear protector and the second ear protector may be separately and securely affixed by an animal handler to the helmet to cover the ears of the animal after the helmet has been secured over the head of the animal.

### BACKGROUND

The present disclosure relates generally to ear protection attachable to helmets. In particular, protective helmets for service and companion animals, namely dogs, are generally, described. A novel attachable ear protector system is then described.

Known animal helmets are not entirely satisfactory for the range of applications in which they are employed. This is particularly surprising as dogs are used in many dangerous activities in ever-increasing numbers. These dangerous activities include sensing bombs or other explosives, searching for contraband by police forces, scrambling over unstable debris after a natural disaster in search of human survivors, and other activities that potentially place the service dog in harm's way.

Companion and service animals are not immune to danger. As the use of animal companions, service animals, and pet ownership continues to rise, more and more of these animals are finding themselves engaging in activities that go well beyond what is typical for a domesticated (albeit highly trained) dog. These dogs can suffer serious injury and even death due to blunt-force trauma that might occur should an accident befall them. Additionally, these activities expose the dog's eyes to flying debris. Thus, there are specially designed helmets with different applications of use and helmets that are uniquely tailored for the type of activity encountered by the animal. These activities, whether military, civilian policing, or adventurous companion dogs, require special fitment for comfort for the dog while providing blunt-force trauma and/or eye protection.

Protective devices, such as a helmet, for both companion and service dogs (canines) have been developed. Such helmets should be comfortable for the dog to wear and further protect the head of the dog from blunt-force trauma to the head and/or the eyes from foreign objects, such as those that might be encountered by service dogs working as law enforcement or military canine units, search and rescue dogs, or for companion dogs that participate in robust exercises and travel with their handlers on motorcycles, all-terrain vehicles, bicycles, and the like.

The known art is all but void of protective gear for the ear area of dogs. Thus, there exists a need for cranial protection systems that improve upon and advance the design of known helmet systems that are also protective of the dog's ears. Examples of new and useful cranial protection relevant to the needs existing in the field are discussed below.

### SUMMARY

The present disclosure is directed to cranial protection systems for terrestrial animals, and in particular, for service dogs. The various embodiments provide a protective helmet that includes ear protection for the dog. An example cranial protection system includes a hard-shell outer layer having pliant panels disposed between the hard-shell components and a soft inner layer, an optional eye protection cover(s), and a set of attachable novel ear covers. In some embodiments, the cranial protection system may be equipped with a communication system that a user may operate to communicate with the service dog wearing the helmet. Additionally, or alternatively, some embodiments may include a noise canceling circuit that eliminates or mitigates ambient noises that would otherwise be heard by the service dog wearing the helmet. Other features and functions will be better appreciated in context with the detailed description and figures.

### BRIEF DESCRIPTION OF THE DRAWINGS

FIG. 1 is a side perspective view of an example embodiment of a cranial protection system prior to attachment of an ear protector(s).

FIG. 2 is front view of the cranial protection system shown in FIG. 1.

FIG. 3 is view of the outside of two ear protectors that are attachably coupled to the helme to cover the ears of the service dog.

FIG. 4 is view of inside of the two ear protectors of FIG. 3.

FIG. 5 is a side perspective view of an example one of the ear protectors.

FIG. 6 is a side view of an embodiment of a cranial protection system with the attached ear protectors.

FIG. 7 is a block diagram showing the electronic circuitry for sound cancellation and/or for wireless communications in an example embodiment of a cranial protection system.

FIG. 8 is a perspective view of the helmet with the ear protectors secured to the head of the service dog.

### DETAILED DESCRIPTION

The disclosed cranial protection system will become better understood through review of the following detailed description in conjunction with the figures. The detailed description and figures provide merely examples of the various inventions described herein. Those skilled in the are will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, examples of various helmets are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example. Other features and functions will be better appreciated in context with the detailed description and figures.

Embodiments are directed to cranial protection systems for terrestrial animals, and in particular, for service dogs. A service dog is a canine that is being controlled in the field to perform a designated task, typically under the supervision of a human handler. The present invention provides a protective helmet for terrestrial animals, namely service dogs and/or other companion animals. Embodiments are ideally suited for animals employed in explosive detection, search and rescue, drug detection, fugitive tracking, accelerant detection, criminal apprehension, search and recovery, military purposes including parachute drops into hostile territory, or other working endeavors. Alternatively, the protective helmet may be used for companion animals during active recreational activities that might include riding in open-top cars, motorcycles, bicycles, all-terrain vehicles or other forms of rough play or recreation. In addition to protecting the head and/or eyes of the service dog wearing the helmet from physical injury, embodiments further protect the ears of the service dog wearing the helmet from physical injury.

An example cranial protection system includes a hard-shell outer layer having pliant panels disposed between the hard-shell components and a soft inner layer, optional eye protection cover(s), and a set of attachable ear covers (interchangeably referred to herein as an ear protector). The ear protector is attachable to the helmet and covers the ears of the service dog wearing the helmet. The ear protector provides protection from blunt-force trauma to the ears and to other nearby areas of the service dog wearing the helmet, and/or provides protection from ear injury that may be caused by loud sounds. Additionally, the service dog wearing the helmet is protected from loud noises, such as exploding ordinance and/or gunfire, which may otherwise damage the service dog's hearing, which may distract the service dog from its current task, and/or which may otherwise disorient the service dog.

Alternatively, or additionally, an ear protector embodiment may facilitate communication of the human handler to the service dog wearing the helmet while blocking out distracting and/or disorienting ambient sounds. In some embodiments, the cranial protection system may be equipped with a communication system that the human handle (interchangeably referred to herein as a “user”) may operate to communicate with the service dog wearing the helmet. Additionally, or alternatively; some embodiments may include a noise canceling circuit that eliminates or mitigates ambient noises that would otherwise be heard by the service dog wearing the helmet.

With reference to FIGS. 1-6, a nonlimiting example cranial protection system 100 (interchangeably referred to herein as a helmet 100) will now be described. Helmet 100 functions to protect a terrestrial animal's head while it is engaged in activities that could place it in harm's way. The reader will appreciate from the figures and description below that helmet 100 addresses shortcomings of conventional cranial protection systems for animals, and in particular service dogs.

For example, helmet 100 contemplates use with different breeds and/or sizes of dogs and different species of animals by being customizable and comprising moving parts that can be fitted to the dog's head for that particular breed and/or dog. Further, helmet 100 is created out of negative space to maximize movement, comfort, and to facilitate the dog's ability to see and hear when the service dog wearing the helmet 100.

FIG. 1 is a side perspective view of an example embodiment of a cranial protection system 100 prior to attachment of the ear protector(s). FIG. 2 is front view of the cranial protection system 100 shown in FIG. 1. As can be seen in FIG. 1, helmet 100 includes a face guard 102, a first side panel 104, a second side panel 106, a rear panel 108, a first lower side panel 110, a second lower side panel 112, a front strap 114, a rear strap 116, a plurality of cushioning means 118, an optional transparent first eye cover 120, an optional transparent second eye cover 122, and an optional supplemental device receiver 128. In an alternative embodiment, the transparent first eye cover 120 and the transparent second eye cover 122 may be a single transparent eye cover that covers both eyes of the service dog wearing the helmet 100.

In a nonlimiting example embodiment, face guard 102, first side panel 104, second side panel 106, and real panel 108 comprise an integrated hard-shell outer affixed to a plurality of cushioning means 180 disposed inside of the helmet 100. Preferably, the face guard 102, the first side panel 104, second side panel 106, and real panel 108 is made of a single fabricated stricture molded to fit the head of the service dog wearing the helmet 100. In alternative embodiments, the face guard 102 is optional and may be removed and attached according to the user's needs and/or the particular breed of service dog wearing the helmet 100.

To facilitate a secure and comfortable fit to the service dog wearing the helmet 100, the lower portion of the first side panel 104 is flexibly coupled to the upper portion of the first lower side panel 110. Similarly, the lower portion of the second side panel 106 is flexibly coupled to the upper portion of the second lower side panel 112. In the various embodiments, a plurality of fasteners 130 that may comprise screws, nails, pegs, brads, and other types of suitable fasteners securely connect the various above-described components of helmet 100 to one another to secure the first lower side panel 110 to the first side panel 104 and to secure the second lower side panel 112 to the first lower side panel 110. However, and suitable attaching means such as straps, fabric, or the like may be used. When the helmet 100 is initially placed onto the head of the service dog, the first lower side panel 110 and the second lower side panel 112 may be flexed outward so that the helmet 100 may be placed onto and over the top of the service dog's head.

The front strap 114, coupled to a leading edge of the first lower side panel 110 and the second lower side panel 112, is secured below the muzzle (chin) of the service dog. The rear strap(s) 116 is affixed to the rear panel 108 and to the rear edges of the first lower side panel 110 and the second lower side panel 112. The front strap 108 and the rear strap 110 cooperatively act together to secure the helmet 100 to the head of the service dog wearing the helmet 100. When the front strap 114 is tighter and secured, the first lower side panel 110 and the second lower side panel 112 are pulled inward, thereby securing the helmet 100 to the head of the service dog wearing the helmet 100. Any suitable strapping means, including buckles, snap clips, Velcro, etc. may be used for the front strap 114 and/or the rear strap 116 in the various embodiments. In some embodiments, the rear strap 116 is permanently attached to the first lower side panel 110 and the second lower side panel 112. The front strap 114 and/or the rear strap 116 may optionally include a length adjustment means may be used to adjust the length of the rear strap(s) 116 to a desired length to better fit the helmet 100 to the head of the service dog wearing the helmet 100. In some embodiments, the straps 114 and/or 116 are made of a stretchable material, such as rubber or the like, that may be stretched to facilitate attachment of the helmet 100 to the service dog, and then retract to maintain tension to secure the helmet 100 to the head of the service dog.

In the various embodiments, the face guard 102, first side panel 104, and second side panel 106 may comprise a solid, inflexible material such as polycarbonate plastic. In other embodiments, the face guard 102, first side panel 104, and second side panel 106 may be made of hard material such as fiberglass, polymers and copolymers, metals and metal alloys, or any other material suitable for providing a protective hard-outer shell. The first lower side panel 110 and the second lower side panel 112 may be made of the same or a similar material. Preferably, the material is impenetrable to objects that may strike the helmet 100 while the service dog is wearing the helmet 100.

In the various embodiments, each of the plurality of cushioning means 118 comprises a soft material such as foam rubber or any soft material suitable to prevent chafing and increase comfort to the wearer, such as cloth, polymer and copolymer foam, or any soft pliant material suitable for such purpose. Further, the plurality of cushioning means 118 provide cushioning and impact protection to the service dog wearing the helmet 100, and facilitate a secure fit to the head of the service dog wearing the helmet 100 after suitable adjustment of the front strap 114 and/or the rear strap 116. In some embodiments, the cushioning means 118 are one or more precut cushion materials that are affixed within the interior of the helmet 100. Alternatively, or additionally, the cushioning means 118 may be made of a spray-on foam and/or a molded cushion. The cushioning means 118 may be permanently attached to the interior of the helmet 100, such as by using an adhesive. Alternatively, the cushioning means 118 may be removable to facilitate cleaning and/or replacement of damaged or work portions of the cushioning means 118.

In the various embodiments, the first side panel 104, second side panel 106, and the rear panel 108 cooperatively act to provide an aperture 132 that is configured to receive one of the ears of the service dog wearing the helmet 100 such that the dog's ears extend outwardly, through the apertures 132 and out from the helmet 100. The aperture 132 is defined by a rim 134. During use, there may be situations wherein the ears of the service dog wearing the helmet 100 does not require ear protection, such as during training and or transport to a work site. Accordingly, the service dog wearing the helmet 100 is able to hear the voice commands of its human trainer and/or hear other ambient noises since its ears are not encased in the ear protectors (described in greater detail herein).

In alternative embodiments, the helmet 100 may be equipped with a variety of accessories that are coupled to the supplemental device receiver 128 depending on the activity, that the animal is engaged in. For example, some embodiments may include equipment capable of sending and/or receiving audiovisual transmissions. For example, a camera may be attached to the supplemental device receiver 128 so that the human handler or another individual may view what the service dog wearing the helmet 100 is seeing. As another non-limiting example, a light may be used to provide light when the service dog wearing the helmet 100 is in a dark enclosure such as a room or a tunnel, or when the service dog wearing the helmet 100 is working at night. Other embodiments, additional equipment such as oxygen masks and the like may be included. In still other embodiments, helmet 100 may include a supplemental visor. In some embodiments, the helmet 100 includes a plurality of supplemental device receivers 128 so that a plurality of devices may be attached to the helmet 100.

FIG. 3 is view of the outside of two ear protectors 302, 304 that are attachable, coupled to the helmet 100 to cover the ears of the service dog. FIG. 4 is view of inside of the two ear protectors 302, 304 of FIG. 3. FIG. 5 is a side perspective view of an example one of the ear protectors 302, 304. FIG. 6 is a side view of an embodiment of a cranial protection system with the attached ear protectors 302, 304. Each one of the ear protectors 302, 304 may be separately and securely affixed to the helmet 100 to cover the ears of the animal after the helmet 100 has been secured over the head of the dog. (Optionally, the ear protectors 302, 304 may be secured to the helmet 100 prior to securing the helmet to the dog's head.)

Each ear protector 302, 304 comprises an edge 306 defining a lower perimeter of the ear protector 302, 304 that is configured to be received into (fit onto) the rim 134 of the aperture 132 of the helmet 100 (FIGS. 1 and 2). Preferably, the edge 306 defines a molded channel that fits into a corresponding rim 134 structure that defines the aperture 132 of the helmet 100 so as to make a gasket like seal between the ear protectors 302, 304 and the helmet 100. Preferably, the seal is soundproof, or substantially sound proof, such that ambient noise is not heard, or is significantly diminished. Any suitable form of the edges 306 of the ear protector 302, 304 and a receiving rim 134 or the aperture 132 may be used in the various embodiments to create the gasket like seal between the ear protectors 302, 304 and the receiving helmet 100. When a molded channel is used to define the edges 306 on the hard material that is sued to fabricate the ear protectors 302, 304, the interaction between the molded channel of the edges 306 and the hard material of the helmet portion that defines the aperture 132 facilitate transfer of forces applied on the ear protectors 302, 304 to the helmet 100. In alternative embodiments, the edges 306 have a molded channel that is configured to receive corresponding edges 306 of the ear protectors 302, 304.

Each ear protector 302, 304 comprises a cup shaped ear cover 308, a lower flange 310 with a stop portion 312, and a latch 314 that engages the corresponding upper edge of the rim 134 of the aperture 132 of the helmet 100 (FIGS. 1 and 2). The lower flange 310 is disposed along a lower portion of the edge 306 of the ear protector 302, 304. The stop portion 312 engages a corresponding lower portion of the rim 134 thereby securing a lower portion of the ear protector 302, 304 to the helmet 100.

The latch 314 is disposed along an upper portion of the edge 306. The latch 324 is located substantially opposite from the lower flange 310 and is configured to engage a corresponding upper portion of the rim 134 of the helmet aperture 132. The latch 324 securely secures a corresponding upper portion of the ear protector 302, 304 to the helmet 100.

In some embodiments, the latch 314 may be opened using an optional latch release button 316 disposed on the outer surface of the ear cover 308. The latch release button 316 extends down through the ear cover 308 so as to engage the latch 314. When engaged with the latch 314, the latch release button 316 pushes the latch 314 away from the rim 132 of the aperture 132 to release the latch 314. Here, when the handler presses down on the latch release button 316 using a single hand, the latch 314 disengages from the rim 134 of the aperture 132 so that the ear protectors 302, 304 can be removed from over the ears of the service dog wearing the helmet 100.

Various types of latches may be used by the various embodiments. One example embodiment employs a latch with no moving parts that is engaged and disengaged from the rim 134 by compressing ear protectors 302, 304, made of a semi-flexible material, in an inward direction. Other embodiments may use a remote control electromechanical latch. Other embodiments may use magnetic latches. The choice of the latch design may be made based upon the anticipate tactical situation that the helmet 100 will be used in.

In an example embodiment, the cushioning means 118 includes a gasket portion 502 (FIG. 5) that extends outwardly beyond the edges 306. In some embodiments, the gasket portion 502 may be configured to be received into the rim 134 of the aperture 132. In some embodiments, the rim 134 may include a molded channel, ledge or other means that is configured to receive and then compress the gasket portion 502 while the ear protectors 302, 304 are secured to the helmet 100. Alternatively, or additionally, the gasket portion 502 may be configured to engage a corresponding portion of the cushioning means 118 that is in the helmet 100 around the aperture 132 and in proximity to the rim 134.

When the ear protectors 302, 304 are securely installed and affixed to the helmet 100, the gasket portion 502 is compressed to create a gasket-like seal between the helmet 100 and the ear protectors 302, 304. Once the ear protectors 302, 304 have been securely attached to the helmet 100 so as to cover the ears of the service dog wearing the helmet 100, the ear protectors 302, 304 become an integral part of the helmet 100. The gasket like seal may, is some embodiments, facilitate the blocking of ambient noises that might otherwise be heard by the service dog wearing the helmet 100.

In an alternative embodiment, an optional rubber gasket or the like may be affixed to either the ear protector 302, 304 and/or around the rim 134 of the aperture 132 of the helmet 100. The gasket may be used to create the gasket-like seal.

In the various embodiments, the cup shaped ear covers 308, interchangeably, referred to herein as an ear pod shell, may comprise a solid, inflexible or semi-flexible material such as polycarbonate plastic. In other embodiments, the cup shaped ear covers 308 may be made of hard material such as fiberglass, polymers and copolymers, metals and metal alloys, or any other material suitable for providing a hard-outer shell. Preferably, the material is impenetrable to objects that may strike the ear protectors 302, 304 while the service dog is wearing the helmet 100. Here, forces applied to the ear protectors 302, 304 that are caused by impacting objects is transferred to the helmet 100 rather than to the head of the service dog wearing the helmet 100.

Preferably, the ear protectors 302, 304 are asymmetrically shaped, being taller in the rear portion to provide adequate interior space for the less flexible part of the dog's ear. The front portion of the cover 308 of the ear protectors 302, 304 may be lower to provide a more streamlined shape with installed on the helmet 100. However, any suitable design of the cover 308 of the ear protectors 302, 304, and/or shape of the base of the ear protectors 302, 304 that is received into the aperture 132, may be used. The shape and size of the cup shaped ear cover 308, and/or shape of the base of the ear protectors 302, 304, may be designed to accommodate the ears of particular breed of dogs. For example, the cup shaped ear covers 308 for a dog with upright rigid ears may be deeper that a cup shaped ear cover 308 for a dog with soft floppy ears. The cup shaped ear covers 308 may be larger for dog breeds having large ears and smaller for dog breeds having smaller ears.

Further, exterior parts of the helmet 100 and the ear protectors 302, 304 may be colored, painted or the like to provide a pleasing appearance to people and/or to facilitate the particular task that the service dog is performing. For example, camouflage may be appropriate in some environment, while black may be suitable for nighttime or other dark environments. Vivid coloring may be desired when the dog is wearing the helmet during recreational activities.

Each ear protector 302, 304 optionally includes a cushioning means 318 disposed on an inner surface of the ear protector 302, 304, In some embodiments, the cushioning means 318 is the same as, or similar to, the plurality of cushioning means 118 disposed on the inside of the helmet 100. In some embodiments, the cushioning means 118 is made of a soft foam or foam-like material that absorbs forces or shocks exerted on the exterior of the ear protectors 302, 304. Accordingly, the cushioning means 318 will cushion the dog's ears in the event that an object impacts the ear protector 302, 304.

In other embodiments, the material of the cushioning means 318 used in the ear protector 302, 304 may be made of an acoustic foam or material that is specially configured to absorb high decibel ambient sounds occurring in an ambient region outside of the helmet 100. When the ear protectors 302, 304 are secured to the helmet 100, the interior region of the helmet 100 is transformed into a “quiet space” much like when the doors of an automobile are closed. Accordingly, loud ambient sounds will not harm the ears, and more particularly the ear drums, of the service dog wearing the helmet 100. Further, it is less likely that the service dog wearing the helmet 100 will be startled, be distracted and/or otherwise become disoriented by any loud or unexpected ambient noises.

In practice, the human handler attaches the ear protectors 302, 304 to the helmet 100 one at a time by inserting each lower flange 310 in through the aperture 132 to engage the lower part of the rim 134. While tucking the dog's ears into the cup shaped ear cover 308, the upper portion of the ear cover 308 is then firmly pressed into the corresponding upper portion of the rim 134 until the latch 314 engages the corresponding upper portion of the rim 134 of the aperture 132. Accordingly, after the latch 314 is securely engaged to the rim 134, the ear protector 302, 304 becomes securely affixed to the helmet 100.

In an example embodiment, to remove the ear protector 302, 304, the human handler pulls the ear protector 302, 304 downward until the latch disengages from the rim 134. In other embodiments, the human handler presses downward on the latch button 316 to disengage the latch 306 from the rim 134 of the aperture 132.

In some embodiments, the two ear protectors 302, 304 are coupled together with an optional length of cord 320. The cord is tethered to the cup shaped ear cover 308 using a molded entry point 322 that is configured to receive and secure an end of the cord 320. The molded entry point 322 is disposed at a selected location on the outside of the ear protectors 302, 304. The length of cord 320 attaching the two ear protectors 302, 304 provides the advantage that the ear protectors 302, 304 always remain tethered together, reducing the likelihood that they will be lost or misplaced by the human handler prior to and/or after use. Additionally, the cord 320 provides the human handler a tactile guide when the ear protectors 302, 304 are attached to the helmet 100 in low lighting conditions or dark environments.

In some embodiments, the cord 320 is made of a stretchable shock cord material that is stretched around and is engaged with securing means 602 (FIG. 6), such as, but not limited to, a knob, hook, pin or the like protruding outward from a backside of the helmet 100, such as on the outside of the rear panel 108 of the helmet 100. When a stretchable shock cord is used, the cord 320 provides tension to the attached ear protectors 302, 304 and the helmet 100 when the cord 320 is engaged with the securing means 602. The tension maintains a seal between the edges of the ear protectors 302, 304 and the rim 134 of the aperture 132 of the helmet 100.

FIG. 6 further conceptually illustrates regions between the helmet 100 and the ear protectors 302, 304 where pressure is exerted when the ear protectors 302, 304 have been secured to the helmet 100. The pressure region 604 is a result of forces exerted by the latch 314 when the latch 314 is engaged with the corresponding portion of the rim 134 or the aperture 132, The pressure region 606 is a result of forces exerted by the cord 320 when the cord 320 is engaged with the corresponding securing means 602 of the helmet 100.

The pressure region 608 is a result of forces exerted by the lower flange 310 and the stop portion 312 when engaged with the corresponding portion of the rim 134 of the aperture 132 at the first lower side panel 110 and the second lower side panel 112, respectively. When the lower flange 310 with the stop portion 312 exerts an outward directed pressure on the first lower side panel 110 and the second lower side panel 112, the front strap 114 and the rear strap 116 cooperatively apply an opposing force. These opposing forces cooperatively act together to maintain a desirable degree of flexibility in the helmet 100 when worn by the service dog.

Some embodiments of the ear protector 302, 304 are provisioned with at least one speaker 324. The speaker 324 is used to generate sound that is then heard by the service dog wearing the helmet 100. Sounds emitted by the speakers 324 may be voice commands from the human handler. Alternatively, or additionally, the sounds emitted by the speaker 324 may be noise cancellation sounds.

Another unexpected advantage of the used of the ear protectors 302, 304 is that they can be securely affixed to the helmet 100 without the use of straps or other complicated fastening means. And, the ear protectors 302, 304 can be attached after the helmet 100 has been secured to the head of the service dog wearing the helmet 100. More particularly, the ear protectors 302, 304 can be attached to the helmet 100 at times when ear protection is desired, and may be removed when ear protection is not needed. Since the human handler can affix the ear protectors 302, 304 one at a time (especially if they are held together by the cord 320) using one hand, the handler's other hand remains free to perform other tasks, such as holding the dog leash, a weapon or other tool.

FIG. 7 is a block diagram showing ear cover electronic circuitry 702 for sound cancellation and/or for wireless communications in an example embodiment of a cranial protection system 100. The ear cover electronic circuitry 702 is integrated into the helmet 100. Components of the ear cover electronic circuitry 702, communicatively coupled together, may be located in any suitable component of the helmet 100.

When used for sound cancellation purposes, the ear cover electronic circuitry 702 comprises a controller 704, a microphone 706, a speaker 324, a processor 708, a memory 710, and a power source 712. Preferably, at least one speaker 324 is included in each one of the ear protectors 302, 304. Memory 710 includes a region for storing the sound amplification or cancellation logic 714. The power source 712 may be any suitable source of power, such as a battery, capacitor, or the like that is used to provide energy to operate the various components of the ear cover electronic circuitry 702. Memory 710 may be any suitable memory medium. Processor 708 may be any suitable device or system that is configured to retrieve and then execute the sound amplification or cancellation logic 714.

One skilled in the art appreciates that from time to time loud ambient noises may occur in the ambient environment in which the service dog wearing the helmet 100 is working in. When such loud noises occur, the service dog may become distracted, and may not be able to perform its current task. Alternatively, or additionally, the loud noise may disorient the service dog, such as when the loud sound is caused by discharge of nearby firearms and/or is caused by a nearby explosion. In other situations, the loud sound may be at a high decibel level that could otherwise damage the service dog's ear drums.

In embodiments provisioned with the sound cancellation feature, one or more microphones 706 disposed on the outside surface of the helmet 100 detect ambient sound. Preferably, the microphone(s) 706 are disposed on the surface of the ear protectors 302, 304. The processor 708, executing the retrieved sound cancellation logic 714, monitors ambient sound detected by the microphone(s) 706. When a loud sound having a decibel level greater than a threshold level is detected by the microphone 706, the processor 708 generates and communicates a sound cancelling signal to the speakers 324. In response to receiving the signal, the speaker 324 generates a sound cancelling noise that cancels out, or at least substantially mitigates, the loud ambient sound so that the service dog wearing the helmet 100 is not distracted, disoriented, and/or harmed by the loud ambient noise.

Alternatively, or additionally, a sound amplification feature may be included. In some situations, such as when the service dog is working in a dark and/or a confined environment where visibility is limited, it may be desirable to amplify ambient sounds so that any muffling effect of the ear protectors 302, 304 are mitigated. Here, sounds detected by the microphones are amplified for reproduction by the speakers 324. Stereoscopic sounds to provide directional cues to the service dog may be provided by including a microphone 706 on either side of the helmet 100 and by reproducing the detected sounds using the corresponding speaker 324.

Alternatively, or additionally, some helmets 100 are embodied with a communication system. Here, a handler's unit 716 is operable to generate and communicate a wireless communication signal to the ear cover electronic circuitry 702. The wireless communication signal includes voice commands spoken by the human handler. The handler's unit 716 comprises a microphone 718, a processor 720, a memory 722, a transceiver 724, and a power source 726. The memory 722 includes a region for storing the analog to digital (A/D) converter logic 728. The power source 726 may be any suitable source of power, such as a battery, capacitor, or the like that is used to provide energy to operate the various components of the handler's unit 716. Memory 722 may be any suitable memory medium. Processor 720 may be any suitable device or system that is configured to retrieve and then execute the A/D converter logic 728.

The microphone 718 detects voice commands being spoken by the human handler. The processor 720, executing the retrieved A/D converter logic 728, converts analog signals corresponding to the sound detected by the microphone 718 into a digital signal. The digital signal is communicated from the processor 720 to the transceiver 724. The transceiver 724 communicates a wireless signal that is received by the transceiver 730 residing in the ear cover electronic circuitry 702. The processor 708, executing the retrieved digital to analog (D/A) converter logic 732, converts the digital information in the received wireless signal into an analog sound signal that is communicated to the speaker 324. The speaker 324, in response to receiving the analog sound signal from the processor 708, then presents the voice commands that are then heard by the service dog wearing the helmet 100.

In an alternative embodiment, the microphone 706 is omitted and the sound cancellation logic 714 resides in the memory 722 of the handler's unit 716. The digital signal communicated from the transceiver 724 to the transceiver 730 in the ear cover electronic circuitry 702 includes the sound cancelling information. Advantageously, the human handler may also be wearing protective ear covering that is also configured to emit sound cancelling noise to protect the human handler.

One skilled in the art appreciates that the sound cancelling feature, the sound amplification feature, and/or the voice command communication feature may be concurrently operating in some embodiments of the helmet 100. In such embodiments, the service dog wearing the helmet 100 can advantageously hear the human handler's voice commands and also not be disturbed, distracted or harmed by loud ambient noises. Such embodiments may be particularly advantageous in battle environment where loud machinery may be operating, fire arms are being discharged, and/or loud explosions occur nearby.

FIG. 8 is a perspective view of the helmet 100 with the ear protectors 302, 304 secured to the head of the service dog 802.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the eight to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A cranial protection system for a terrestrial animal, the protection system comprising:
a helmet that is secured to the head over ears of the animal,
wherein the helmet includes a first aperture and a second aperture that are each configured to receive the one of the ears of the animal when the helmet has been secured to the animal's head, and
wherein each aperture is defined by a rim about the helmet aperture; and
a first ear protector and a second ear protector; wherein each ear protector comprises:
a cup shaped ear cover that fits over one of the ears of the animal when the ear protector is secured to the helmet;
an edge that defines a lower perimeter of the ear protector, wherein the edge is configured to be received into the rim of the helmet aperture when the ear protector is secured to the helmet;
a lower flange with a stop portion disposed along a lower portion of the edge of the ear protector, wherein the lower flange engages a corresponding lower portion of the rim of the helmet aperture, and wherein the stop portion secures the lower flange to the helmet; and
a latch disposed along an upper portion of the edge of the ear protector, wherein the latch is located substantially opposite from the lower flange and is configured to engage a corresponding upper portion of the rim of the helmet aperture, and wherein the latch securely secures a corresponding upper portion of the ear protector to the helmet,
wherein each one of the first ear protector and the second ear protector may be separately and securely affixed by an animal handler to the helmet to cover the ears of the animal after the helmet has been secured over the head of the animal.

2. The cranial protection system of claim 1, wherein at least one of the first ear protector and the second ear protector comprises:
a cushioning means disposed on an inner surface of the ear protector.

3. The cranial protection system of claim 2, wherein at cushioning means is made of an acoustic foam or material that absorbs high decibel ambient sounds occurring in an ambient region outside of the helmet.

4. The cranial protection system of claim 2, wherein at cushioning means is made of a material that absorbs forces or shocks exerted on the exterior of the ear protectors.

5. The cranial protection system of claim 1, wherein each ear protector comprises;
a molded entry point disposed on the outside of the ear cover, wherein the molded entry point is configured to receive and secure an end of a cord and
the cord, wherein the cord tethers the first ear protector and the second ear protector together.

6. The cranial protection system of claim 5, wherein the cord is made of a stretchable shock cord material, and wherein helmet comprises:
a securing means that protrudes outward from a backside portion of the helmet,
wherein when the cord is engaged with the securing means after the ear protectors have been secured to the helmet, the cord provides a tension force to the ear protectors and the helmet.

7. The cranial protection system of claim 1, wherein at least one of the first ear protector and the second ear protector comprises:
a latch release button that extends down through the ear cover 308,
wherein the latch 314 latch release button, when pressed downward by the animal handler, engages the latch 314 to push the latch 314 away from the rim of the helmet aperture to release the latch.

8. The cranial protection system of claim 1, further comprising:
at least one microphone disposed on the outside of the helmet or on the outside of at least one of the first ear protector and the second ear protector, wherein the at least one microphone detects ambient sounds;
a speaker disposed on an inside of at least one of the first ear protector and the second ear protector;
a memory that stores sound cancellation logic; and
a processor that is communicatively coupled to the at least one microphone, the speaker, and the memory,
wherein the processor monitors the ambient sounds detected e at least one microphone, and
wherein in response to the detected sound has a decibel level greater than a threshold level, the processor generates a sound cancelling signal that is communicated to the speaker,
wherein the speaker generates a cancelling sound that mitigates the detected sound heard by the animal through the ear protectors.

9. The cranial protection system of claim 1, further comprising:
at least one microphone disposed on the outside of the helmet or on the outside of at least one of the first ear protector and the second ear protector, wherein the at least one microphone detects ambient sounds;
a speaker disposed on an inside of at least one of the first ear protector and the second ear protector;
a memory that stores sound amplification logic; and
a processor that is communicatively coupled to the at least one microphone, the speaker, and the memory, wherein the processor monitors the ambient sounds detected by the at least one microphone,
wherein the speaker generates an amplification sound that amplifies the detected sound heard by the animal through the ear protectors.

10. The cranial protection system of claim 1, further comprising:
a transceiver configured to receive a wireless signal containing voice commands them the animal handler;
a speaker disposed on an inside of at least one of the first ear protector and the second ear protector;
a memory that stores digital to analog converter logic; and
a processor that is communicatively coupled to the transceiver, the speaker, and the memory,
wherein the processor receives information corresponding to the wireless signal received by the transceiver, and
wherein in the processor, executing the digital analog converter logic, generates an analog sound signal that is communicated to the speaker,
wherein the speaker, in response to receiving the analog sound signal from the processor, presents sound corresponding to the voice commands spoken by the animal handler.
